(19) United States
(12) Patent Application Publication
(10) Pub. No.: US 2022/0313994 A1
(43) Pub. Date: Oct. 6, 2022

(54) NON-INVASIVE BRAIN STIMULATION HEALTH CARE DEVICE

(71) Applicant: NEUROEN LTD., Wonju-si, Gangwon-do (KR)

(72) Inventors: Do Yeun OH, Wonju-si, Gangwon-do (KR); Jae Won LEE, Seocho-gu, Seoul (KR); Min Soo KIM, Seoul (KR); Kyung Soo LEE, Yeongdeungpo-gu, Seoul (KR)

(73) Assignee: NEUROEN LTD., Wonju-si, Gangwon-do (KR)

(21) Appl. No.: 17/636,914

(22) PCT Filed: Apr. 19, 2021
(86) PCT No.: PCT/KR2021/004893

§ 371 Date: Feb. 21, 2022

(30) Foreign Application Priority Data
Apr. 21, 2020 (KR) 10-2020-0048016
Feb. 15, 2021 (KR) 10-2021-0019603
Feb. 15, 2021 (KR) 10-2021-0019604

(51) Int. Cl. A61N 1/36; A61N 1/04; A61N 1/20

(52) U.S. Cl. CPC A61N 1/36025; A61N 1/0456; A61N 1/36031; A61N 1/20

(57) ABSTRACT

An embodiment discloses a healthcare device for non-invasive brain stimulation, including a plurality of electrode units that applies electrical stimulation to a brain of a subject; a power supply unit that supplies power to the plurality of electrode units; and a control unit that controls the electrical stimulation applied to the brain of the subject by controlling the plurality of electrode units and the power supply unit, wherein the electrical stimulation includes a buffer mode using tACS and a main mode using tDCS.

### TECHNICAL FIELD

The embodiment relates to a healthcare device for non-invasive brain stimulation.

### BACKGROUND ART

Mild cognitive impairment refers to a state in which memory or other cognitive functions are clearly diminished enough to be confirmed by objective tests, but the ability to carry out daily life is preserved and is not yet dementia. However, mild cognitive impairment is a high-risk group for dementia. In the case of normal elderly, the rate of progression to dementia is about 1 to 2% each year, but it is known that about 10 to 15% of people with mild cognitive impairment progress to dementia every year. Mild cognitive impairment belongs to the high-risk group for dementia, with about 80% progressing to dementia after 6 years.

As treatment methods for mild cognitive impairment, there are drug therapy, cognitive therapy, and brain stimulation therapy.

As drug treatment, Alzheimer's disease treatment, ginkgo leaf extract, choline precursor, vitamin B group supplement, etc. can be used, and if depression is the cause, antidepressants are sometimes used. However, there are no drugs that have been proven to treat mild cognitive impairment, and above all, the risk of drug misuse is very high.

Cognitive therapy is for a cognitive rehabilitation for the brain function of a degraded area, and corresponds to a treatment for improving cognitive deficits and improving social function using scientific learning principles. However, cognitive therapy has a problem that it takes a long time to improve and the effect is weak.

Brain stimulation treatment includes invasive treatment such as deep brain stimulation and non-invasive treatment that stimulates the brain from the outside with magnetism, electricity, or ultrasound. However, the invasive treatment has a risk of side effects and medical complications after surgery, so research on non-invasive treatment is being actively conducted.

Non-invasive treatment of mild cognitive impairment does not show immediate effect compared to invasive treatment, but has the advantage of fewer side effects and a significant effect compared to cognitive treatment.

A representative example of non-invasive treatment is a method using transcranial current stimulation (tCS), and specifically, a method of activating a specific part of the subject's brain or causing rest with transcranial direct current stimulation (tDCS) is widely used.

The treatment using tDCS is less effective when the subject's tension or stress is high. Conventionally, in order to increase the effect of tDCS, an attempt has been made to lower the subject's tension or stress by playing music, etc., but there is a problem that the effect varies greatly from person to person.

In addition, tDCS causes discomfort such as a stinging pain in the skin at the beginning of the procedure, and this discomfort lowers the periodic and long-term availability of the subject's healthcare system, and even if used, there is a problem of increasing the subject's tension or stress.

In addition, in order to increase the effectiveness of non-invasive treatment, periodic and continuous treatment is required. However, it is practically impossible for the subject to receive treatment more than once or twice a day at a hospital. In addition, the method of using electrical stimulation, which is a representative example of non-invasive treatment of mild cognitive impairment, is to apply electrical stimulation by attaching electrodes to the scalp. Electrical stimulation devices available for conventional mild cognitive impairment have a problem that it is too difficult for ordinary people to wear. For example, there are problems in that it is difficult to position the plurality of electrodes of the electrical stimulation device in an appropriate position, and the adhesion of the electrodes is prevented by hair.

After all, in order to improve the mild cognitive impairment of the subject by using electrical stimulation, which is a non-invasive treatment, a new device that can be used easily and conveniently by the subject is needed.

### DETAILED DESCRIPTION OF INVENTION

### Technical Problem

An embodiment provides a healthcare device for brain stimulation that can prevent and effectively treat mild cognitive impairment.

An embodiment provides a healthcare device for non-invasive brain stimulation with improved electrode adhesion and wearability that can be directly worn by a subject.

An embodiment provides a healthcare system for non-invasive brain stimulation that can further improve the treatment effect by lowering the subject's tension or stress.

On the other hand, other objects not specified in the present invention may be additionally considered within a range that can be easily inferred from the following detailed description and effects thereof.

### Solution to Problem

A healthcare device for non-invasive brain stimulation according to an embodiment of the present invention for solving the above-described problem includes: a plurality of electrode units that applies electrical stimulation to a brain of a subject; a power supply unit that supplies power to the plurality of electrode units; and a control unit that controls the electrical stimulation applied to the brain of the subject by controlling the plurality of electrode units and the power supply unit, wherein the electrical stimulation includes a buffer mode using tACS and a main mode using tDCS.

The buffer mode may be performed before the main mode is performed.

A sensor unit that measures electrocardiogram of the subject may be further included.

When the buffer mode is performed, the control unit collects information on the electrocardiogram of the subject from the sensor unit, and the control unit analyzes the information on the electrocardiogram of the subject and terminates the buffer mode when the subject is in a normal state to perform the main mode.

The plurality of electrode units may include a first electrode unit that applies the electrical stimulation to a left frontal lobe of the brain of the subject; a second electrode unit that applies the electrical stimulation to a right frontal lobe of the brain of the subject; a third electrode unit that applies the electrical stimulation to a left parietal lobe of the brain of the subject; a fourth electrode unit that applies the electrical stimulation to a right parietal lobe of the brain of the subject; and a fifth electrode unit that is disposed on a head of the subject and has a polarity different from the polarity of the first to fourth electrode units.

The device includes a helmet that has a dome shape so as to be worn on the subject's head and in which the plurality of electrode units is disposed; and an upper electrode adhesion module that is installed on the helmet. The upper electrode adhesion module includes a first band adjusting unit that is fixedly installed on the helmet; and a first band of which length is adjusted by the first band adjusting unit, and that is configured as a ring to surround the head of the subject the inside of the helmet. At least some of the plurality of electrode units are installed on the first band, and when the length of the first band is shortened by the first band adjusting unit, the electrode unit installed in the first band may be in close contact with the frontal lobe of the subject.

At least some of the plurality of electrode units are installed on the inner rear side of the helmet, and when the length of the first band is shortened by the first band adjusting unit, the electrode unit installed on the inner rear side of the helmet may support in close contact with a region including at least a portion of the parietal lobe, occipital lobe, and temporal lobe of the subject.

Some other of the plurality of electrode units may be installed at a location capable of adhering to a region of the first band including at least a portion of the parietal, occipital, and temporal lobes of the subject.

The device further includes a lower electrode adhesion module that is installed on the helmet. The lower electrode adhesion module includes a second band adjusting unit that is installed independently of the helmet; and a second band of which length is adjusted by the second band adjusting unit and that is configured to partially cover the back of the neck or the back of the ear of the subject. Some of the plurality of electrode units are installed on the second band, when the length of the second band is shortened by the second band adjusting unit, the electrode unit installed in the second band may be in close contact with a region including at least a portion of the back of the ear, the back of the head, and the back of the neck of the subject.

The device further includes a sensor unit that is installed on the second band, and when the length of the second band is shortened by the second band adjusting unit, the sensor unit may be in close contact with a region including at least a part of the back of the ear, the back of the head, and the back of the neck of the subject.

The electrode units include a wet pad, and the helmet includes a first frame that is formed to cover from the forehead to the back of the head, and has an opening at the upper portion thereof, and a second frame that extends upward from the first frame and crosses over the head of the subject, and humidity or moisture generated in the wet pad may be discharged through the opening.

An LED monitoring unit that displays an operating state of the electrode units may be installed on the helmet.

### Advantageous Effects of Invention

According to an embodiment, since mild cognitive impairment can be prevented by a device mounted on an equipment such as a helmet, the subject can safely perform brain stimulation.

In addition, since it can be manipulated to operate according to a predetermined program, there is an effect that the subject can safely and conveniently perform brain stimulation.

In addition, in the form of a helmet worn on the head of the subject, the subject can easily wear it alone.

In addition, the subject wears a helmet and manipulates the first dial in a state in which the third electrode unit and the fourth electrode unit are in close contact with a region including at least a portion of the parietal, occipital, and temporal lobe sides to bring the first electrode unit and the second electrode unit into close contact with the frontal lobe, and manipulates the second dial to bring the fifth electrode unit into close contact with a region including at least a portion of the back of the ear, the back of the head, and the back of the neck.

On the other hand, in the helmet, the portion where the first to fourth electrode units are installed is opened, so that the moisture evaporation problem caused by the use of the wet electrode can be solved.

In addition, by operating the buffer mode using tACS before the main mode using tDCS (concentration improvement mode, memory improvement mode, etc.), it is possible to further improve the treatment effect of the main mode by lowering the subject's tension and stress before receiving the main mode treatment.

In addition, the healthcare system for invasive brain stimulation according to an embodiment of the present invention can reduce the pain caused by skin stimulation of tDCS by operating the buffer mode before the start of the main mode or between consecutive main modes.

It will be apparent that even if effects not explicitly mentioned herein, the effects described in the following specification expected by the technical features of the present invention and their provisional effects are treated as if they were described in the specification of the present invention.

### BRIEF DESCRIPTION OF DRAWINGS

FIG. 1 is a schematic structural view of a healthcare system according to an embodiment of the present invention.

FIGS. 2 to 5 are schematic reference views for explaining various electrical stimulation of a healthcare system according to an embodiment of the present invention.

FIG. 6 is a view schematically illustrating the form of electrical stimulation of tDCS and tACS.

FIG. 7 is a reference view for explaining a check mode, a buffer mode, and a main mode of a healthcare system according to an embodiment of the present invention.

FIG. 8 is a schematic reference view for illustrating an example of a program of a healthcare system according to an embodiment of the present invention.

FIGS. 9 to 11 are quantitative EEG data before and after the procedure of a healthcare system according to an embodiment of the present invention.

FIG. 12 is a schematic side view of a healthcare device in which a healthcare system according to an embodiment of the present invention is implemented.

FIG. 13 is a schematic rear view of a healthcare device according to another embodiment of the present invention.

FIG. 14 is a schematic bottom view of a healthcare device according to another embodiment of the present invention.

FIG. 15a is a schematic plan view of an upper electrode adhesion module of a healthcare device according to another embodiment of the present invention.

FIG. 15b is a schematic perspective view of an inside of a helmet of a healthcare device according to another embodiment of the present invention.

FIG. 16 is a schematic plan view of a lower electrode adhesion module of a healthcare device according to another embodiment of the present invention.

It should be understood that the accompanying drawings are exemplified by reference for understanding the technical idea of the present invention, and the scope of the present invention is not limited thereby.

### EMBODIMENTS OF INVENTION

Hereinafter, the configuration of the present invention guided by various embodiments of the present invention and effects resulting from the configuration will be described with reference to the drawings. In the description of the present invention, if it is determined that related known functions are obvious to those skilled in the art and may unnecessarily obscure the gist of the present invention, the detailed description thereof will be omitted.

As used herein, the term “module” may include a unit implemented in hardware, software, or firmware, and may be used interchangeably with terms such as, for example, logic, logic block, component, or circuit. A module may be an integrally formed component or a minimum unit or a part of the component that performs one or more functions.

In this document, a “module” or “node” performs operations such as moving, storing, and converting data by using an arithmetic device such as a CPU or AP. For example, a “module” or “node” may be implemented as a device such as a server, PC, tablet PC, smartphone, and the like.

A Healthcare System for Non-Invasive Brain Stimulation

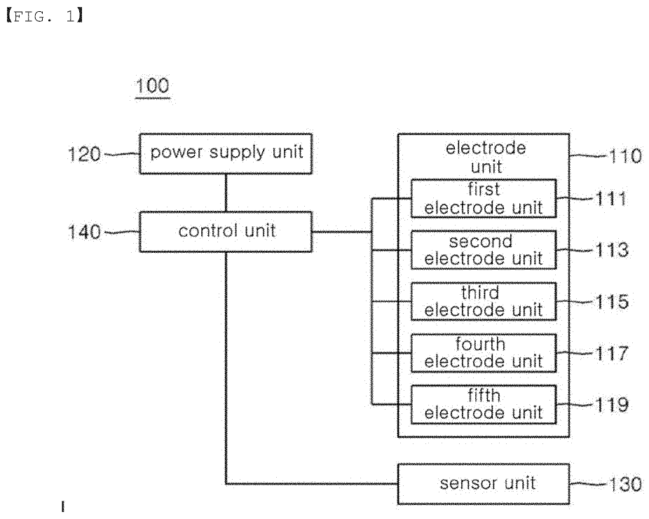

FIG. 1 is a schematic structural view of a healthcare system for non-invasive brain stimulation (hereinafter, referred to as a “healthcare system”) according to an embodiment of the present invention.

Hereinafter, a healthcare system (device) according to an embodiment of the present invention will be described with reference to the drawings.

A healthcare system 100 according to an embodiment of the present invention is configured to non-invasively apply electrical stimulation through an electrode in close contact with the scalp of a subject. Through such electrical stimulation, various brain-caused diseases can be prevented, treated, and managed. For example, using the healthcare system 100 according to an embodiment of the present invention, not only mild cognitive impairment but also depression, convulsive disease, pain, memory improvement, motor learning ability improvement, intellectual disability, addiction disease, schizophrenia, etc. can be prevented, treated and managed.

The healthcare system 100 according to an embodiment of the present invention includes a plurality of electrode units 110, a power supply unit 120, a sensor unit 130, and a control unit 140. On the other hand, the healthcare system 100 of the present invention is implemented by the healthcare system for non-invasive brain stimulation to be described later, so that a subject can easily use the healthcare system 100. A healthcare device for non-invasive brain stimulation according to another embodiment of the present invention will be described in detail later.

The plurality of electrode units 110 is in close contact with various positions of the subject's head. The plurality of electrode units 110 may receive power from the power supply unit 120 to allow current to flow. Some of the plurality of electrode units 110 may be positive electrodes, and others may be negative electrodes. In addition, it is also possible that current flows only in some of the plurality of electrode units 110 and no current flows in some of the plurality of electrode units. That is, the polarity or operation of each electrode unit may be changed according to a program provided by the healthcare system 100 according to an embodiment of the present invention.

The first electrode unit 111 is in close contact with a position corresponding to the left frontal lobe of the subject's head, the second electrode unit 113 is in close contact with a position corresponding to the right frontal lobe of the subject's head, the third electrode unit 115 is in close contact with a position corresponding to a region including at least a portion of the left parietal lobe, left occipital lobe, and left temporal lobe of the subject's head, and the fourth electrode unit 117 is in contact with a position corresponding to a region including at least a portion of the right parietal lobe, right occipital lobe, and right temporal lobe of the subject's head. In the first to fourth electrode units 111, 113, 115, and 117, current may flow by the power supplied from the power supply unit 120. In this case, the first to fourth electrode units 111, 113, 115, and 117 may be either a positive electrode through which current is emitted or a negative electrode through which current is received.

The fifth electrode unit 119 may be in close contact with the subject's head at a position different from the positions of the first to fourth electrode units 111, 113, 115, and 117, and may be in close contact with a region including at least a portion of, for example, the back of ears, the back of the head, and the back of the neck of the subject. In this case, the fifth electrode unit 119 may be either a positive electrode through which current is emitted or a negative electrode through which current is received.

For the first electrode unit 111, the second electrode unit 113, the third electrode unit 115, the fourth electrode unit 117, and the fifth electrode unit 119, an electrode containing moisture may be used. However, the present invention is not limited thereto, and the first electrode unit 111, the second electrode unit 113, the third electrode unit 115, the fourth electrode unit 117, and the fifth electrode unit 119 may include a material to which current can be constantly transmitted without moisture, if necessary.

In addition, although the present embodiment is described as simulating the frontal lobe and the parietal lobe using the first electrode unit 111, the second electrode unit 113, the third electrode unit 115, the fourth electrode unit 117, and the fifth electrode unit 119, it is not limited thereto. The first electrode unit 111, the second electrode unit 113, the third electrode unit 115, the fourth electrode unit 117, and the fifth electrode unit 119 may be arrange to apply electrical stimulation to other portions of the brain.

The electrical stimulation applied to each electrode unit 110 may be controlled by the control unit 140. In the healthcare system 100 according to an embodiment of the present invention, the control unit 140 may control so that a transcranial current stimulation (tCS) is applied to the electrode unit 110. As the type of transcranial current stimulation used in the present invention, at least one of transcranial direct current stimulation (tDCS), transcranial alternating current stimulation (tACS), and transcranial random-noise stimulation (tRNS) or a combination thereof may be used. In particular, in the healthcare system 100 according to an embodiment of the present invention, when the subject wears the healthcare device and starts a healthcare operation, the control unit 140 first performs tACS, and then can perform tDCS when the condition of the subject becomes a normal state. With such a configuration, it is possible to further increase the treatment efficiency for the subject. This will be described later.

The power supply unit 120 may supply the power required for the operation of the healthcare system 100 according to an embodiment of the present invention through the power used.

The power supply unit 120 may supply power such that the first electrode unit 111, the second electrode unit 113, the third electrode unit 115, and the fourth electrode unit 117 are positive electrodes, and the fifth electrode unit 119 is a negative electrode. Alternatively, the power supply unit 120 may supply power such that the first electrode unit 111, the second electrode unit 113, the third electrode unit 115, and the fourth electrode unit 117 are negative electrodes, and the fifth electrode unit 119 is a positive electrode. In addition, in a state in which power is not supplied to the third electrode unit 115 and the fourth electrode unit 117, the power supply unit 120 may supply power such that the first electrode unit 111 and the second electrode unit 113 are positive electrodes, and the fifth electrode unit 119 is a negative electrode. In this way, the power supplied from the power supply unit 120 to the first electrode unit 111, the second electrode unit 113, the third electrode unit 115, the fourth electrode unit 117, and the fifth electrode unit 119 may be controlled by the control unit 140.

The power supply unit 120 may adjust an amount of current of power supplied to the first electrode unit 111, the second electrode unit 113, the third electrode unit 115, the fourth electrode unit 117, and the fifth electrode unit 119. In this embodiment, the current flowing through each of the first electrode unit 111, the second electrode unit 113, the third electrode unit 115, the fourth electrode unit 117, and the fifth electrode unit 119 may be about 0.5 mA.

The power supply unit 120 may supply power to the first electrode unit 111, the second electrode unit 113, the third electrode unit 115, the fourth electrode unit 117, and the fifth electrode unit 119 through a commercial power supply, and may be a battery if necessary. When the power supply unit 120 is a battery, a rechargeable secondary battery may be used.

The healthcare system 100 according to an embodiment of the present invention may further include a sensor unit 130 that measures various states of a user. Like the fifth electrode unit 119, the sensor unit 130 may be in close contact with the subject's head at a position different from the positions of the first to fourth electrode units 111, 113, 115 and 117, for example, it may be in close contact with a region including at least a portion of the back of the ear, the back of the head, and the back of the neck of the subject. When the sensor unit is closely attached to the back of the ear, the fifth electrode is preferably located on the right side and the sensor unit is preferably located on the left side for more effective heart rate measurement. If the present healthcare system does not include the sensor unit, a sixth electrode unit may be included at the position of the sensor unit, instead of the sensor unit, and the sixth electrode unit performs the same role as the fifth electrode unit.

The sensor unit 130 may be an electrocardiogram sensor unit for measuring an electrocardiogram of a subject. In this case, the electrocardiogram sensor unit collects human body information of the subject, and checks whether the current applied through the electrode unit 110 causes a problem in the human body. In particular, in the healthcare system 100 according to an embodiment of the present invention, when the subject wears the healthcare device and starts the healthcare procedure, the control unit 140 may be configured to first perform tACS, and to perform tDCS after the condition of the subject becomes normal. The sensor unit of electrocardiogram serves to determine whether the state of the subject is normal. Whether the condition of the subject is normal is determined by using the collected information on the electrocardiogram in a comfortable condition for the subject, or commonly known information on the electrocardiogram in the normal state (for example, resting heart rate by age, etc.).

The control unit 140 controls the current flowing through the first electrode unit 111, the second electrode unit 113, the third electrode unit 115, the fourth electrode unit 117, and the fifth electrode unit 119. In this case, the control unit 140 controls so that the power is supplied to only some of the first electrode unit 111, the second electrode unit 113, the third electrode unit 115, the fourth electrode unit 117, and the fifth electrode unit 119. In addition, the control unit can control so that each can be independently driven as a positive electrode or a negative electrode.

The control unit 140 may control the intensity of the current flowing through the first electrode unit 111, the second electrode unit 113, the third electrode unit 115, and the fourth electrode unit 117 and the fifth electrode unit 119 according to the user's selection. For example, the control unit 140 may control the current flowing through the first electrode unit 111, the second electrode unit 113, the third electrode unit 115, and the fourth electrode unit 117 including the wet or dry pad unit in the range of 0.1 mA to 5 mA. For example, the control unit 140 may adjust the current in units of 0.5 mA. In addition, the control unit 140 may automatically adjust the intensity of the current according to the mode, if necessary.

The control unit 140 receives the human body information of the subject transmitted from the sensor unit 130. The control unit 140 may store the received human body information of the subject. Meanwhile, the control unit 140 may analyze the received human body information and change the type or intensity of the electrical stimulation transmitted to the electrode unit.

Meanwhile, the control unit 140 may further include a communication module (e.g., WIFI, Bluetooth, etc.). When the control unit 140 includes the communication module, it is possible to transmit information on the operation of the healthcare system 100, including human body information of the subject, to a terminal (e.g., a smartphone, computer, etc.) of the subject. In addition, the information on the mode or the number of times the subject uses the healthcare system 100 may be transmitted. Accordingly, the subject may receive a guide on the use of the healthcare system 100 and may receive management of the product.

The control unit 140 stores various types of electrical stimulation according to the symptoms of the subject.

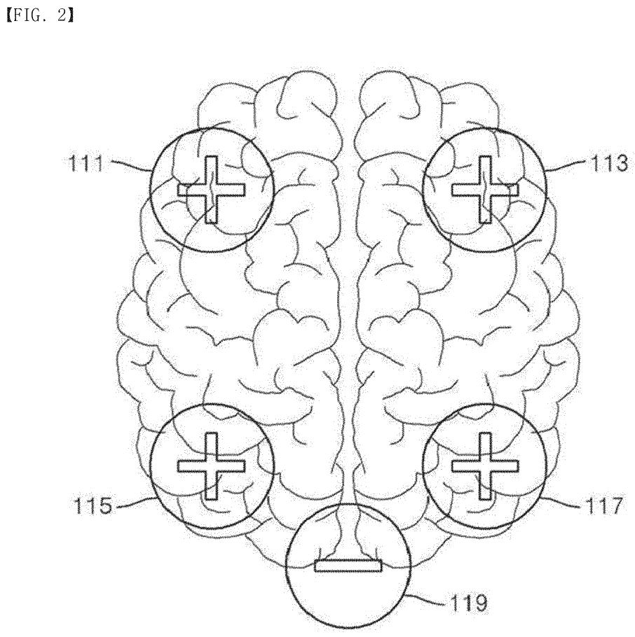

FIGS. 2 to 5 are schematic reference views for explaining various electrical stimulation of a healthcare system according to an embodiment of the present invention.

FIG. 2 is a reference view for explaining the electrical stimulation of the simultaneous improvement mode of memory and concentration.

To improve memory and concentration at the same time, it is necessary to stimulate and activate the frontal lobe in charge of memory and the parietal lobe in charge of concentration at the same time. Therefore, in the simultaneous improvement mode of memory and concentration, the control unit 140 controls the first electrode unit 111, the second electrode unit 113, the third electrode unit 115, and the fourth electrode unit 117 to be driven as positive electrodes, and the fifth electrode unit 119 to be driven as a negative electrode. If there is no sensor unit, a sixth electrode unit may be included at a position of the sensor unit, instead of the sensor unit, and the sixth electrode unit may be controlled to be driven by a negative electrode in the same way as the fifth electrode unit.

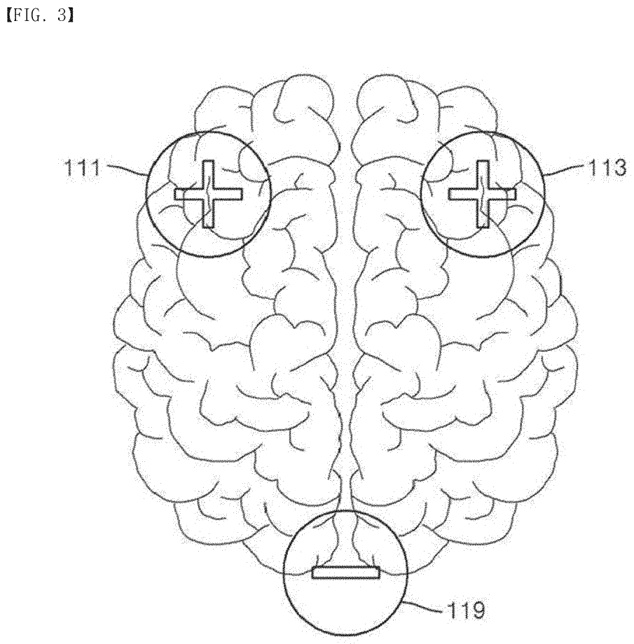

FIG. 3 is a reference view for explaining electrical stimulation in a memory improvement mode.

In order to improve memory, it is necessary to stimulate and activate the frontal lobe in charge of memory. Accordingly, in the memory improvement mode, the control unit 140 may control the first electrode unit 111 and the second electrode unit 113 to be driven as a positive electrode and the fifth electrode unit 119 to be driven as a negative electrode. In this case, the third electrode unit 115 and the fourth electrode unit 117 may be controlled not to operate. If there is no sensor unit, a sixth electrode unit may be included at a position of the sensor unit instead of the sensor unit, and the sixth electrode unit may be controlled to be driven as a negative electrode in the same way as the fifth electrode unit.

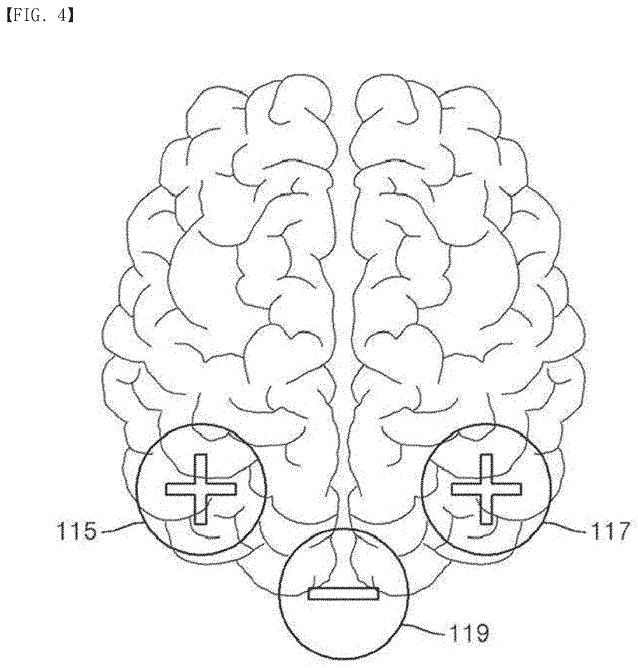

FIG. 4 is a reference view for explaining electrical stimulation in a concentration improvement mode.

In order to improve concentration, it is necessary to stimulate and activate the parietal lobe in charge of concentration. Therefore, in the concentration improvement mode, the control unit 140 may control the third electrode unit 115 and the fourth electrode unit 117 to be driven as positive electrodes and the fifth electrode unit 119 to be driven as a negative electrode. In this case, the first electrode unit 111 and the second electrode unit 113 may be controlled not to operate. If there is no sensor unit, a sixth electrode unit may be included at a position of the sensor unit instead of the sensor unit, and the sixth electrode unit may be controlled to be driven by a negative electrode in the same way as the fifth electrode unit.

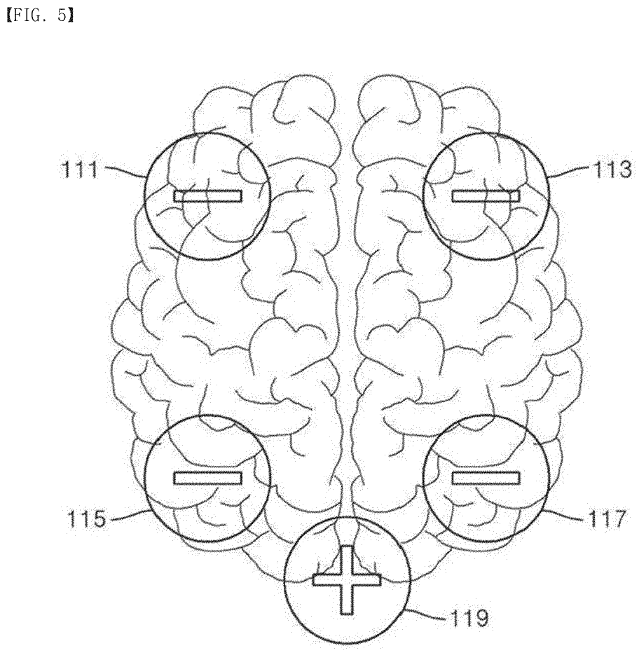

FIG. 5 is a reference view for explaining electrical stimulation in a sleep mode (insomnia improvement mode).

In order to induce sleep, the brain needs to be stimulated to rest as a whole. Accordingly, in the sleep mode, the control unit 140 may control the first electrode unit 111, the second electrode unit 113, the third electrode unit 115, and the fourth electrode unit 117 to be driven as negative electrodes, and the fifth electrode unit 119 to be driven as a positive electrode. If there is no sensor unit, a sixth electrode unit may be included at a position of the sensor unit instead of the sensor unit, and the sixth electrode unit may be controlled to be driven by a negative electrode in the same way as the fifth electrode unit. The ‘insomnia mode’ is a mode that can be used when to rest the brain.

In addition to the above modes, at least one of the first electrode unit 111, the second electrode unit 113, the third electrode unit 115, and the fourth electrode unit 115 may be driven to stimulate at least one of the frontal and parietal lobes in order to improve speech ability, spatiotemporal ability, emotion regulation, and auditory ability.

In addition, the control unit 140 may block the current flowing through the first electrode unit 111, the second electrode unit 113, the third electrode unit 115, the fourth electrode unit 117, and the fifth electrode unit 119 according to the user's electrocardiogram measured by the sensor unit 130. For example, when the user's electrocardiogram measured by the sensor unit 130 is out of the normal range, the control unit 140 may block the current flowing through the first electrode unit 111, the second electrode unit 113, the third electrode unit 115, the fourth electrode unit and the fifth electrode unit 119.

Meanwhile, in each of the modes of FIGS. 2 to 5, electrical stimulation may be applied as tDCS. In addition, the modes shown in FIGS. 2 to 5 may be combined to form a program.

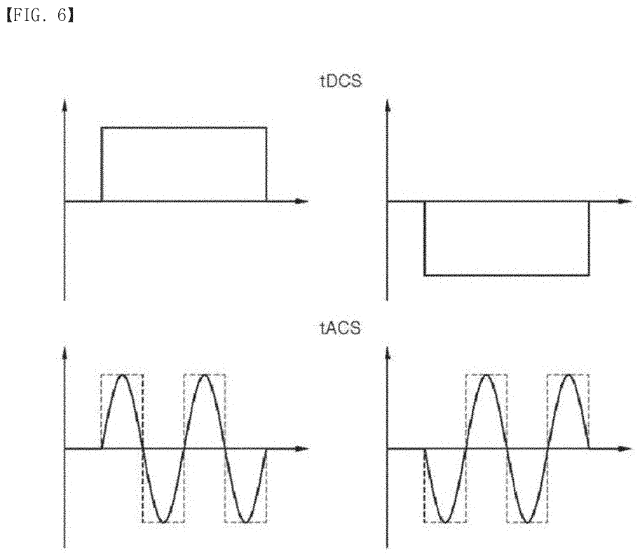

FIG. 6 is a view schematically illustrating the form of electrical stimulation of tDCS and tACS.

In the case of tDCS, the stimulation of the same polarity is generally applied for several minutes by using direct current. In contrast, in the case of tACS, the stimulation of a rectangular (dotted line) or sinusoidal (solid line) is applied by using alternating current.

tDCS regulates spontaneous neural activity in the brain through polarized electrical stimulation. For example, it is effective in the regulation of decision-making, memory, language, sensory perception, etc. for each portion of the brain. In contrast, since tACS uses the alternating current, it is practically impossible to control the directionality (e.g., upward or downward) of electric current in brain regions. Therefore, tDCS is more widely used than tACS for the prevention, treatment and management of depression, convulsive disease, pain, intellectual disability, addictive disease and mild cognitive impairment, and for effects such as memory improvement and motor learning ability improvement. However, it is known that tDCS has a problem in that the treatment effect decreases due to fatigue or stress of the subject. In addition, tDCS causes discomfort such as a stinging pain in the skin at the beginning of the procedure, and this discomfort lowers the periodic and long-term availability of the subject's healthcare system, and even if used, there is a problem that increases the stress of the subject. In particular, when cognitive decline occurs, the subject's anxiety and tension increase, and the discomfort of tDCS further increases the subject's anxiety and tension.

The healthcare system 100 according to an embodiment of the present invention proposes a program configured to solve the above-described problems of tDCS.

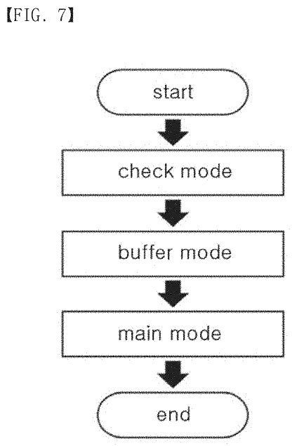

FIG. 7 is a reference view for explaining a check mode, a buffer mode, and a main mode of a healthcare system according to an embodiment of the present invention.

When the subject selects any one of the programs provided by the healthcare system, the control unit 140 first performs a check mode. In the check mode, it is checked whether the subject properly wears the healthcare device, which will be described later. In the check mode, the resistance of the electrode unit 110 is measured, and an appropriate action is guided to the subject according to the range of the resistance value. For example, when the impedance of the electrode unit is lower than the installed load, it can be determined as normal, when the impedance of the electrode unit is in the range of 1-2 times that of the load, it can be determined that the wet pad lacks moisture, and when the impedance of the electrode unit exceeds twice that of the load, it can be determined as poor adhesion of the electrode unit.

When it is determined that all the electrode units 110 are normal, a buffer mode is performed. The buffer mode uses tACS to allow the skin in contact with the electrode unit to adapt to stimulation, thereby lowering skin stimulation caused by tDCS in the following main mode. For tACS, the electrical stimulation of 4 to 40 Hz can be used. Preferably, the buffer mode (mode 0) may use tACS (alpha). tACS (alpha) refers to the electrical stimulation of 8 to 12 Hz, and has the effect of relieving the subject's tension. Therefore, if the buffer mode is performed first and then the main mode is performed, the treatment effect of the main mode is further increased.

The main mode consists of at least one of a memory improvement mode, a concentration improvement mode, a simultaneous improvement mode of memory and concentration, and a sleep mode. The main mode consists of ramp up-stimulation-ramp down. By locating the ramp-up step in the first half of the stimulation of the main mode, it gives enough time for the skin of the subject to adapt to the stimulation, thereby reducing discomfort.

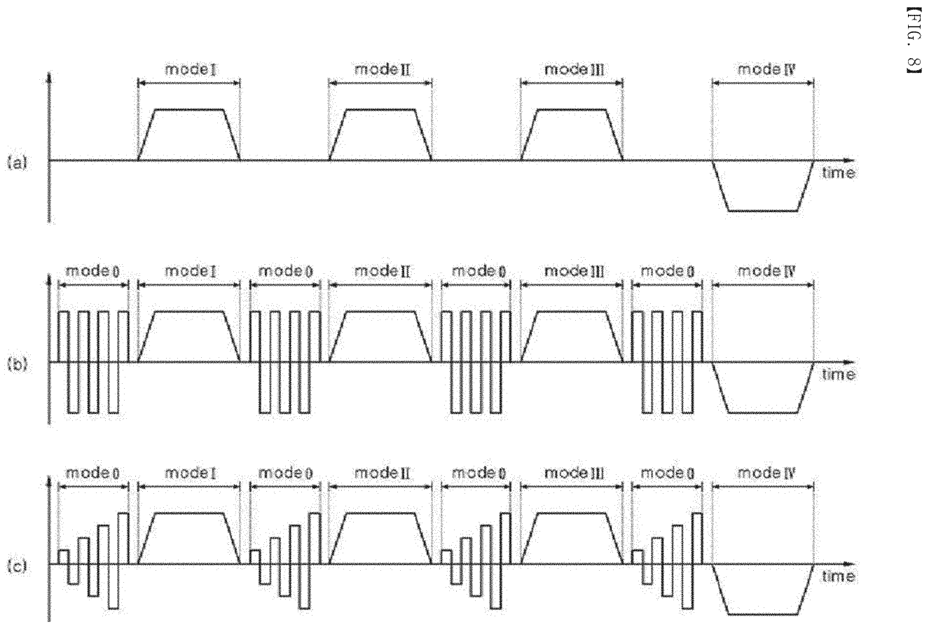

FIG. 8 is a schematic reference view illustrating various examples of programs of the healthcare system 100 according to an embodiment of the present invention.

In FIG. 8, (a) shows a program in which a memory improvement mode (mode I), a concentration improvement mode (mode II), a simultaneous improvement mode of memory and concentration (mode III) are sequentially performed, and finally, a sleep mode (mode IV) is performed.

In FIG. 8, (b) and (c), as in (a), are a program in which the memory improvement mode (mode I), the concentration improvement mode (mode II), the simultaneous improvement mode of memory and concentration (mode III) are sequentially performed, and finally the sleep mode (mode IV) is performed.

In the case of (b) and (c) in FIG. 8, the buffer mode (mode 0) is performed using tACS at the beginning of the program.

When the main mode consists of two or more modes selected from the memory improvement mode (mode I), the concentration improvement mode (mode II), the simultaneous improvement mode of memory and concentration (mode III), and the sleep mode (mode IV), the buffer mode (mode 0) is located between each of the modes, and it may serve to relieve tension of the subject.

On the other hand, when the electrode unit is a wet pad, the impedance may increase due to the evaporation of moisture from the wet pad into body temperature during use. Accordingly, even while the main mode is being performed, the control unit 140 may periodically determine whether the electrode unit is abnormal in the same manner as in the check mode.

The control unit 140 may measure the change in electrocardiogram of the subject according to the execution of the buffer mode (mode 0) by using the sensor unit 130. In the main mode using tDCS, the subject's tension increases due to pain caused by electrical stimulation, and accordingly, changes in the electrocardiogram occur. The control unit 140 may start a scheduled main mode operation when it is determined that the electrocardiogram is in a normal state by sensing the change in electrocardiogram of the subject while operating the buffer mode (mode 0). When the electrocardiogram is in a normal state, it means the measurement value of electrocardiogram in a comfortable state of the subject, and it can be determined based on the heart rate or rhythm.

Each mode composing the program is usually performed for 10 to 30 minutes, but, if necessary, it can be performed for less than 1 hour, and the intensity is determined within −5 mA to +5 mA. On the other hand, in the case of tACS, the amplitude can be controlled within −1 mA to +1 mA.

On the other hand, as shown in (c) of FIG. 8, the buffer mode (mode 0) allows the subject's skin to more smoothly adapt to the stimulus by gradually increasing the amplitude as time goes by.

Table 1 below is a summary of the results of a survey for the subjects to be treated after the procedure according to each program of FIG. 8. A comparative example is a survey result for 100 people who used the healthcare system according to the program of (a) in FIG. 8. Example 1 is a survey result for 100 people who used the healthcare system according to the program of (b) of FIG. 8, and Example 2 is a survey result for 100 people who used the healthcare system according to the program of (c) of FIG. 8.

TABLE 1

| | Percentage of subjects who | Degree of |
|---|---|---|
| | felt pain | satisfaction |
| Comparative | 82% | 81% |
| Example | | |
| Example 1 | 23% | 97% |
| Example 2 | 8% | 98% |

Referring to Table 1, the rate of feeling discomfort such as stinging pain at the beginning of the program operation of the healthcare system 100 according to an embodiment of the present invention is 82% in Comparative Example, 23% in Example 1, only 8% in Example 2. In particular, in the case of Example 2, it was answered that the degree of pain was very mild. In the case of satisfaction with the healthcare system according to an embodiment of the present invention, all of them showed relatively high satisfaction, but in the cases of Examples 1 and 2, in which the degree of pain was mild, higher satisfaction was shown.

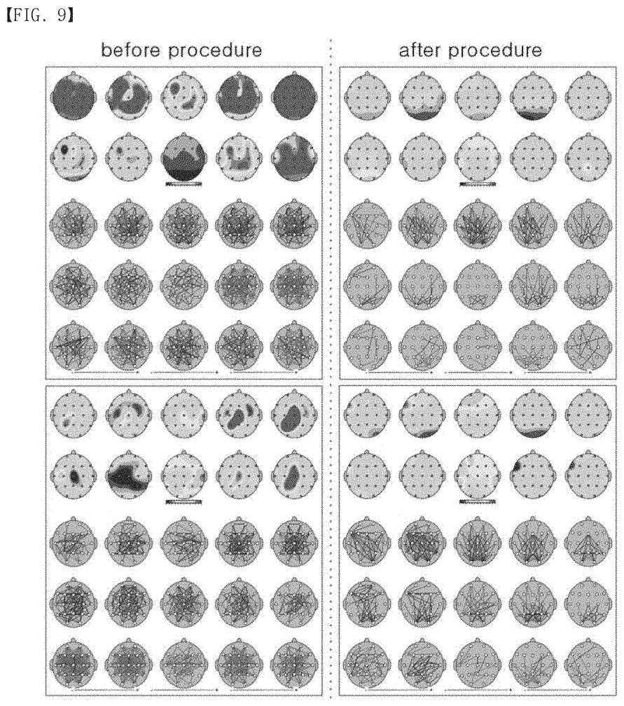

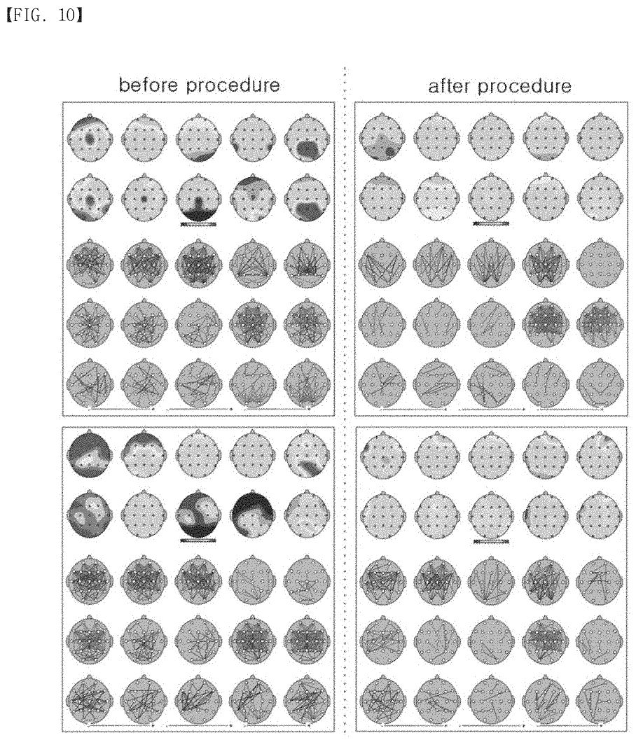

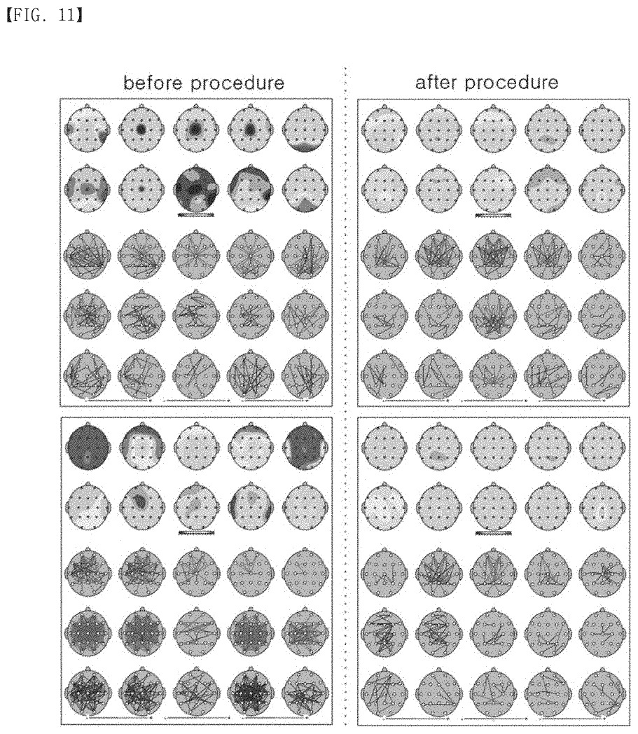

FIGS. 9 to 11 are quantitative EEG (electroencephalogram) data before and after the procedure of the healthcare system according to an embodiment of the present invention. FIG. 9 is a quantitative EGG data of a subject who is addicted to games at a high level before and after the procedure, FIG. 10 is a quantitative EEG data of a subject with tic disorder and ADHD symptoms before and after the procedure, and FIG. 11 is a quantitative EEG data of a subject receiving Asperger's speech therapy before and after the procedure. In FIGS. 9 to 11, it was checked that abnormal EEG (dark color) appeared before the procedure, and normal EEG (light color) was restored after the procedure.

The healthcare system for non-invasive brain stimulation as described above may be implemented as a program (or application) including an executable algorithm that can be executed on a computer. The program may be provided by being stored in a non-transitory computer readable medium.

Here, the non-transitory readable medium refers to a medium that stores data semi-permanently and can be read by a device, not a medium that stores data for a short moment, such as a register, cache, memory, etc. Specifically, various applications or programs described above may be provided by being stored in the non-transitory readable medium such as a CD, DVD, hard disk, Blu-ray disk, USB, memory card, ROM, and the like.

A Healthcare Device for Non-Invasive Brain Stimulation

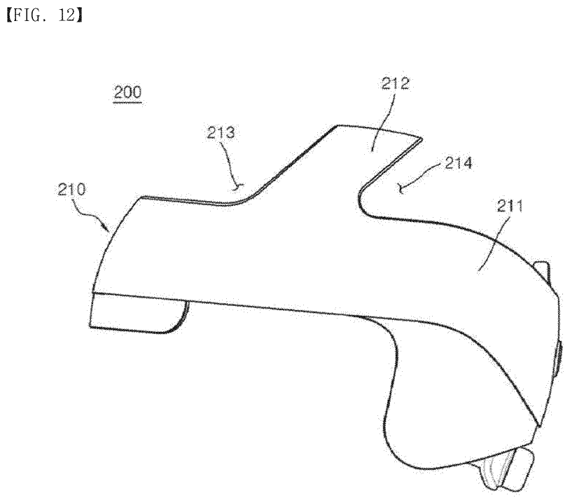

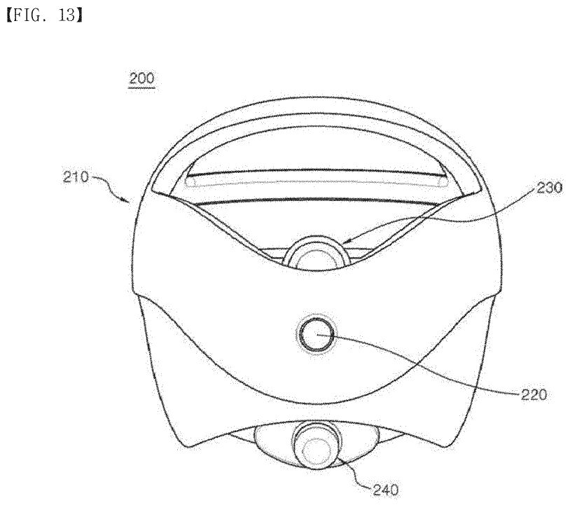

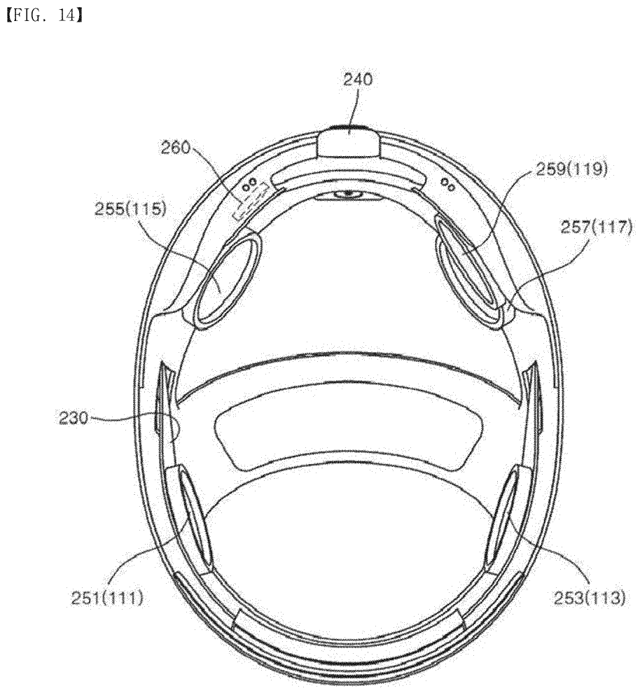

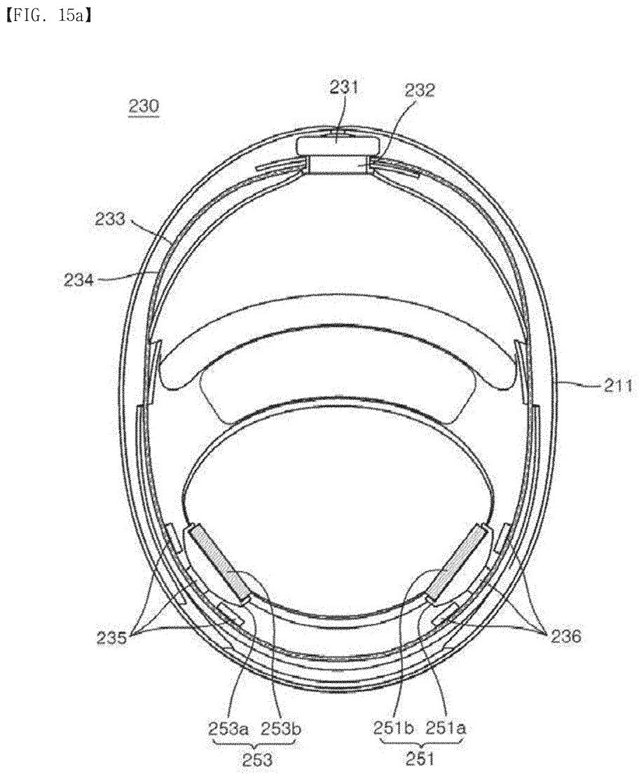

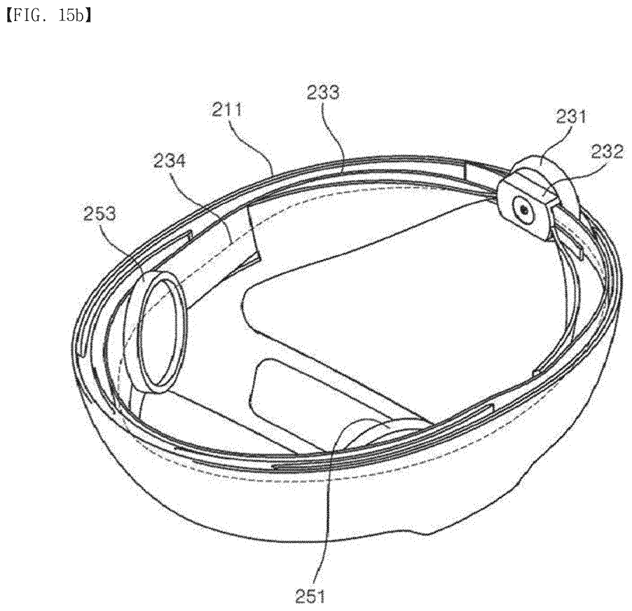

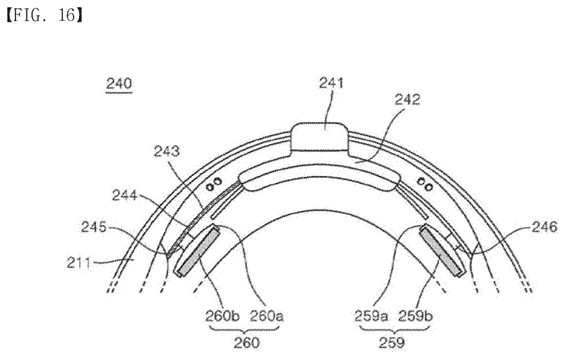

FIG. 12 is a schematic side view of a healthcare device in which a healthcare system according to an embodiment of the present invention is implemented, FIG. 13 is a schematic rear view of a healthcare device according to another embodiment of the present invention, and FIG. 14 is a schematic bottom view of a healthcare device according to another embodiment. In addition, FIG. 15 (a) is a schematic plan view of an upper electrode adhesion module of a healthcare device according to another embodiment of the present invention, FIG. 15 (b) is a schematic perspective view of an inside of a helmet of a healthcare device according to another embodiment of the present invention, and FIG. 16 is a schematic plan view of a lower electrode adhesion module of a healthcare device according to another embodiment of the present invention.

Hereinafter, a healthcare device according to another embodiment of the present invention will be described with reference to the drawings.

A healthcare device according to another embodiment of the present invention includes a helmet 210 having a dome shape so as to be worn on the head of a subject, and a plurality of electrode units 251, 253, 255, 257, 259 installed inside the helmet 210. On the other hand, when the healthcare device according to another embodiment of the present invention does not have a sensor unit, an additional electrode unit may be further included.

The aforementioned power supply unit 120, the sensor unit 130, and the control unit 140 may also be mounted on the helmet 210, and an adjusting unit through which the user can adjust the healthcare system 100 for the mild cognitive impairment prevention can be installed to the outer surface of the helmet 210.

Accordingly, when the user wears the helmet 210 and selects a mode to be used, the user can use the helmet 210 for a set time (e.g., about 20 minutes at a time).

The helmet 210 is composed of a first frame 211 that is formed to cover from the forehead to the back of the head of the subject, who is wearer, and has openings 213 and 214 on an upper portion thereof, and a second frame 212 that extends upward from the first frame 211 to cross the top of the head of the subject. The opening of the upper portion of the first frame 211 is divided into a first opening 213 and a second opening 214 by the second frame 212.

The power supply unit 120 and the control unit 140 of the healthcare system 100 according to an embodiment are installed inside the frames 211 and 212 of the helmet 210. A button 220 is installed on the outer surface of the helmet 210 to control the power and operation of the healthcare system 100. On the other hand, when the control unit 140 includes a communication module, it is also possible for the subject to control the healthcare system 100 using his own smartphone and the like.

The helmet 210 may further include a short-range communication module such as Bluetooth capable of performing short-range wireless communication with, for example, a smart phone or tablet PC used by the user. Therefore, the user can check and control the operation of the healthcare system for mild cognitive impairment prevention using a smartphone or tablet PC.

In addition, an external communication module capable of accessing the Internet and the like may be installed in the helmet 210. Therefore, the information on the electrocardiogram measured by the sensor unit 130 can be transmitted to the hospital used by the user through the Internet network, and information on the mode or number of times the user uses the healthcare system for mild cognitive impairment prevention can be transmitted. Accordingly, the user may receive guidance on the use of the healthcare system for mild cognitive impairment prevention, and may receive management of the product.

As in FIG. 14, a plurality of electrode units 251, 253, 255, 257 and 259 is installed inside the helmet 210. In order to operate the mode or program of the healthcare system 100 on the subject using the healthcare device 200 according to another embodiment of the present invention, the electrode unit must be in close contact with the subject's head.

To this end, the healthcare device 200 according to another embodiment of the present invention includes an upper electrode adhesion module 230 and a lower electrode adhesion module 240 installed on the helmet 210.

The upper electrode adhesion module 230 includes a first band adjusting unit 232 installed with a fixed position on the helmet 210, a first band 233 having a length adjusted by the first band adjusting unit 232, and a first electrode unit 251 and a second electrode unit 253 installed on the first band 233. The first band 233 constitutes a ring along the head of the subject on the inside of the helmet 210.

The length of the first band 233 may be adjusted by manipulating the first dial 231 of the first band adjusting unit 232. For example, when the first dial 231 is turned clockwise (or counterclockwise), the length of the first band 233 is shortened, and when the first dial 231 is turned counterclockwise (or clockwise), the length of the first band 233 is increased. For example, there are gears and teeth rotated by the first dial 231 inside the first band adjusting unit 232, and the length can be adjusted by pushing or pulling the first band 233 while the teeth rotate. However, the present invention is not limited thereto, and it is also possible to apply another method (e.g., an electric motor) capable of adjusting the length of the first band 233.

A first electrode socket 235 and a second electrode socket 236 are installed on the first band 233. At least one electric wire 234 is built in the first band 233, and the electric wire is connected to the first electrode socket 235 and the second electrode socket 236, respectively. The first electrode unit 251 is connected to the first electrode socket 235, and the second electrode unit 253 is connected to the second electrode socket 236. The first electrode socket 235 and the second electrode socket 236 may be configured in plurality, respectively, and the subject may install the first electrode unit 251 and the second electrode unit 253 in the first electrode socket 235 and the second electrode socket located at positions suitable for his head.

On the other hand, the third electrode unit 255 and the fourth electrode unit 257 are installed inside the helmet 210, that is, on the back of the head (near the parietal lobe) of the subject. The third electrode unit 255 and the fourth electrode unit 257 also have a plurality of sockets (not shown) installed inside the helmet 210 like the first electrode unit 251 and the second electrode unit 253, and the third electrode unit 255 and the fourth electrode unit 257 are installed at appropriate positions among the sockets. Alternatively, the third electrode unit 255 and the fourth electrode unit 257 may be installed at a position that can be in close contact with a region of the first band 233 including at least some of the parietal, occipital, and temporal lobes of the subject. In this case, an additional band adjusting unit capable of adjusting the length of the first band may be installed in the middle of the first band (between the first electrode unit and the third electrode unit, and between the second electrode unit and the fourth electrode unit). The additional band adjusting unit may use a known technique for increasing or decreasing the length of the band.

The subject wears the helmet 210 in a state where all the electrodes are installed inside the helmet and the first band 233 is lengthened. Then, when the subject reduces the length of the first band 233 by manipulating the first dial, the first band 233 is shorten in a state where the third electrode unit 255 and the fourth electrode unit 257 support the back head of the subject, the first electrode unit 251 and the second electrode unit 253 are in close contact with the frontal part of the subject, that is, the frontal lobe part of the subject.

The subject inserts the electrode unit into the socket at an appropriate location and manipulates the first dial, thereby easily attaching the first electrode unit 251, the second electrode unit 253, the third electrode unit 255, and the fourth electrode unit 257 to the head.

The first electrode unit 251 may be composed of a pad receiving part 251a and a wet pad 251b, or may use a dry pad formed of a multilayer hydrogel composite. Such a configuration may be similarly applied to the second to fifth electrode units 253, 255, 257 and 259. Here, the first electrode unit 251 will be described as an example.

When the first electrode unit 251 is composed of the pad receiving part 251a and the wet pad 251b, the subject uses the pad 251b of sponge material after soaking it in water. This is the same for other electrode units other than the first electrode unit 251. Therefore, when using the healthcare device 200 according to another embodiment of the present invention, moisture is evaporated by body temperature, and when the helmet 210 is sealed, the subject's discomfort increases due to the moisture, which causes odor when used for a long time. The healthcare device 200 according to another embodiment of the present invention forms the first opening 213 and the second opening 214 on the upper portion of the helmet 210, thereby discharging moisture or water vapor generated during use to the outside so that discomfort and odor problems of the subject due to moisture can be prevented in advance.

The lower electrode adhesion module 240 includes a second band adjusting unit 242 installed independently of the helmet 210, a second band 243 having a length adjusted by the second band adjusting unit 242, and a fifth electrode unit 259 installed at the second band 243. In addition, a sensor unit 260 may be further installed on the second band 243. The second band 243 is configured to partially cover a region including at least a portion of the back of the ear, the back of the head, and the back of the neck of the subject. If the present healthcare system does not include the sensor unit, a sixth electrode unit may be included at the position of the sensor unit, instead of the sensor unit, and the sixth electrode unit performs the same role as the fifth electrode unit.

The end of the second band 243 is fixed to the helmet 210. The length of the second band 243 may be adjusted by manipulating the second dial 241 of the second band adjusting unit 242. For example, when the second dial 241 is turned clockwise (or counterclockwise), the length of the second band 243 is shortened, and when the second dial 241 is turned counterclockwise (or clockwise), the length of the second band 243 is increased. For example, there are gears and teeth rotated by the second dial 241 inside the second band adjusting unit 242, and the length can be adjusted by pushing or pulling the second band 243 while the teeth rotate. However, the present invention is not limited thereto, and it is also possible to apply another method (e.g., an electric motor) capable of adjusting the length of the second band 243.

A fifth electrode socket 245 is installed on the second band 243. In addition, the sensor unit socket 246 may be further installed in the second band 243. At least one electric wire 244 is built in the second band 243, and the electric wire is connected to the fifth electrode socket 245 and the sensor unit socket 246, respectively. The fifth electrode unit 259 is connected to the fifth electrode socket 245, and the sensor unit 260 is connected to the sensor unit socket 246. The fifth electrode socket 245 and the sensor unit socket 246 may be configured in plurality, respectively, and the subject may install the fifth electrode unit 259 and the sensor unit 260 on the fifth electrode socket 245 and the sensor unit socket 246 at positions suitable for his head.

After the first to fourth electrode units 251, 253, 255, 257 are in close contact using the upper electrode adhesion module 230, the subject manipulates the second dial 241 of the lower electrode adhesion module 240 to reduce the length of the second band 243. Then, as the length of the second band 243 is reduced, the fifth electrode unit 259 or the sensor unit 260 is configured to partially cover a region including at least a portion of the back of the ear, the back of the head, and the back of the neck of the subject. As the sensor unit, it is also possible to use a sensor unit of a known electrocardiogram sensor.

Meanwhile, the helmet 210 may further include an LED monitoring unit (not shown). The LED monitoring unit is composed of 5 or 6 LEDs, and serves to display the operating status of the electrode unit or the sensor unit (whether the current value/resistance value is satisfied, etc.).

On the other hand, an antifouling coating layer made of an antifouling coating composition may be applied to the outer surface of the electrode unit to effectively prevent adhesion of pollutants and remove pollutants.

The antifouling coating composition contains mercaptobenzothiazole and amidoalkyl betaine in a molar ratio of 1:0.01 to 1:2, and the total content of mercaptobenzothiazole and amidoalkylbetaine is 1 to 10% by weight based on the entire aqueous solution.

The molar ratio of mercaptobenzothiazole and amidoalkyl betaine is preferably 1:0.01 to 1:2. When the molar ratio is out of the above range, the applicability of the substrate decreases or moisture adsorption on the surface increases after coating, so that there is a problem in that the coating layer is removed.

The mercaptobenzothiazole and amidoalkyl betaine are preferably 1 to 10% by weight in the entire aqueous solution of the composition. If it is less than 1% by weight, there is a problem in that the applicability of the substrate is reduced, and if it exceeds 10% by weight, crystal precipitation is likely to occur due to an increase in the thickness of the coating layer.

On the other hand, as a method of applying the present antifouling coating composition on a substrate, it is preferable to apply by a spray method. In addition, the thickness of the final coating layer on the substrate is preferably 550 to 2000 Å, more preferably 1100 to 1900 Å. If the thickness of the coating layer is less than 550 Å, there is a problem of deterioration in the case of high-temperature heat treatment, and if it exceeds 2000 Å, there is a disadvantage in that crystal precipitation on the coated surface is easy to occur.

In addition, the present antifouling coating composition may be prepared by adding 0.1 mol of mercaptobenzothiazole and 0.05 mol of amidoalkyl betaine to 1000 ml of distilled water and then stirring.

In addition, as a fragrance material having a sterilizing function is coated on the inner and outer surfaces of the helmet 210, it effectively sterilizes (stress relief) the inner and outer surfaces of the helmet 210.

On the other hand, functional oil may be mixed with the fragrance material, and the mixing ratio is 95 to 97% by weight of the functional oil and 3 to 5% by weight of the functional oil, and the functional oil consists of 50% by weight of costus oil and 50% by weight of cassie oil.

Here, the functional oil is preferably mixed in an amount of 3 to 5% by weight based on the fragrance. When the mixing ratio of the functional oil is less than 3% by weight, the effect is insignificant, and when the mixing ratio of the functional oil exceeds 3 to 5% by weight, the effect is not significantly improved while the manufacturing cost is greatly increased.

Costus oil has good effects on neuralgia, muscle pain, antidepression, stress relief, and the like.

Cassie oil is effective in detoxifying, treating skin diseases, sterilizing, relieving itching, clearing the head, relieving tension, and the like.

As this functional oil is coated on the inner and outer surfaces of the helmet 210, an excellent effect of sterilizing the inner and outer surfaces of the helmet 210 is exhibited. The reason for limiting to the above components and the numerical values of the ratios is that, as a result of analysis through the test results while repeating failure several times by the present inventor, the optimal effect is shown in the components and ratios.

In addition, an adhesion enhancer is applied to improve adhesion when the first to fifth electrode units 111, 113, 115, 117, and 119 are attached to the inner surface of the helmet 210, and the adhesion enhancer may be composed by containing 53 parts by weight of water, 15 parts by weight of ethyl acrylate, 20 parts by weight of butyl acrylate, 3 parts by weight of toyltriazole, 5 parts by weight of a surfactant, 1 part by weight of an additive, 2 parts by weight of ammonium persulfate, and 1 part by weight of a buffer.

Here, when the water solvent is added in an amount of less than 53 parts by weight based on 100 parts by weight of the total, the overall concentration increases and the spray operation becomes difficult. When the water solvent is added in an amount of 53 parts by weight or more, the concentration is thin and spraying is easy but dripping occurs. Thus, it is preferable to add the water in an amount of 53 parts by weight.

When the ethyl acrylate is added in an amount of 15 parts by weight based on 100 parts by weight of the total, flexibility and water resistance are lowered, so it is not easy to mix with the water solvent. When the ethyl acrylate is added in an amount of 15 parts by weight or more, flexibility and water resistance are increased, but it becomes a factor of an increase in overall viscosity and makes spraying difficult. Thus, it is preferable to add the ethyl acrylate in an amount of 15 parts by weight.

When the butyl acrylate is added in an amount of less than 20 parts by weight based on 100 parts by weight of the total, adhesiveness, water resistance, and gloss properties are lowered, so that it cannot function as an adhesion enhancer between the first to fifth electrode units 111, 113, 115, 117, 119 and the inner surface of the helmet 210. When the butyl acrylate is added in an amount of 20 parts by weight or more based on 100 parts by weight of the total, water resistance, glossiness, and adhesiveness are increased, but it becomes a factor of an increase in the overall viscosity, making spraying difficult. Thus, it is preferable to add the butyl acrylate in an amount of 20 parts by weight.

The surfactant uses an anionic type and a nonionic type together and uses a commonly known conventional material that is uniformly mixed by dispersing a monomer in water. When the surfactant is added in an amount of less than 5 parts by weight based on 100 parts by weight of the total, the hydrophilicity and lipophilicity between the water solvent and the monomer are lowered, mixing and homogenization are difficult. When the surfactant is added in an amount of 5 parts by weight or more based on 100 parts by weight of the total, the effect of hydrophilicity and lipophilicity between the water solvent and the monomer can no longer be expected. Thus, it is preferable to add the surfactant in an amount of 5 parts by weight.

The additive is a general stabilizer that helps to maintain the continuous physical properties of the product, and is preferably added in an amount of 1 part by weight.

When the ammonium persulfate, which is catalyst, is added in an amount of less than 2 parts by weight based on 100 parts by weight of the total, the reaction among the monomers does not occur smoothly, so it is difficult to obtain good physical properties. When the ammonium persulfate is added in an amount of 2 parts by weight or more based on 100 parts by weight of the total, this becomes a factor of causing the deterioration of physical properties. Thus, it is preferable to add the ammonium persulfate in an amount of 2 parts by weight.

The buffer is preferably added in an amount of 1 part by weight based on 100 parts by weight of the total that does not affect the overall physical properties and viscosity.

When the reaction step is completed, an aging step for about 90 minutes in a state in which the temperature of the reactor is maintained at 72° C. is performed, and then a cooling step at room temperature is performed. Accordingly, the manufacturing process of the adhesion enhancer that enhances the adhesion between the first to fifth electrode units 111, 113, 115, 117, 119 and the inner surface of the helmet 210 according to the present invention is completed.

Water, which is used as a solvent of an adhesion enhancer for improving adhesion between the first to fifth electrode units 111, 113, 115, 117, 119 and the inner surface of the helmet 210 according to the present invention made as described above, is contained in an amount of 53 parts by weight, and thus, the concentration is low, and it is possible to apply it to the inner surface of the helmet 210 by a spray method.

The reason for limiting to the above components and their numerical values is that, as a result of several tests by the present inventor, a remarkable operating effect is shown under the conditions as described above.

The protection scope of the present invention is not limited to the description and expression of the embodiments explicitly described above. In addition, since obvious changes or substitutions are possible in the technical field to which the present invention pertains, it is mentioned once again that the protection scope of the present invention cannot be limited.

1. A healthcare device for non-invasive brain stimulation, comprising:
a plurality of electrode units that applies electrical stimulation to a brain of a subject;
a power supply unit that supplies power to the plurality of electrode units; and
a control unit that controls the electrical stimulation applied to the brain of the subject by controlling the plurality of electrode units and the power supply unit,
wherein the electrical stimulation includes a buffer mode using tACS and a main mode using tDCS.

2. The healthcare device for non-invasive brain stimulation according to claim 1, wherein the buffer mode is performed before the main mode is performed.

3. The healthcare device for non-invasive brain stimulation according to claim 1, further comprising a sensor unit that measures electrocardiogram of the subject.

4. The healthcare device for non-invasive brain stimulation according to claim 3, wherein when the buffer mode is performed, the control unit collects information on electrocardiogram of the subject from the sensor unit, and the control unit analyzes the information on the electrocardiogram of the subject and terminates the buffer mode when the subject is in a normal state to perform the main mode.

5. The healthcare device for non-invasive brain stimulation according to claim 1, wherein the plurality of electrode units includes:
a first electrode unit that applies the electrical stimulation to a left frontal lobe of the brain of the subject;
a second electrode unit that applies the electrical stimulation to a right frontal lobe of the brain of the subject;
a third electrode unit that applies the electrical stimulation to a left parietal lobe of the brain of the subject;
a fourth electrode unit that applies the electrical stimulation to a right parietal lobe of the brain of the subject; and
a fifth electrode unit that is disposed on a head of the subject and has a polarity different from the polarity of the first to fourth electrode units.

6. The healthcare device for non-invasive brain stimulation according to claim 1, further comprising:
a helmet that has a dome shape so as to be worn on the subject's head and in which the plurality of electrode units is disposed; and
an upper electrode adhesion module that is installed on the helmet,
wherein the upper electrode adhesion module includes:
a first band adjusting unit that is fixedly installed on the helmet; and
a first band of which length is adjusted by the first band adjusting unit, and that is configured as a ring to surround the head of the subject the inside of the helmet,
wherein at least some of the plurality of electrode units are installed on the first band, and when the length of the first band is shortened by the first band adjusting unit, the electrode unit installed in the first band is in close contact with a frontal lobe of the subject.

7. The healthcare device for non-invasive brain stimulation according to claim 6, wherein at least some of the plurality of electrode units are installed on an inner rear side of the helmet, and when the length of the first band is shortened by the first band adjusting unit, the electrode unit installed on the inner rear side of the helmet supports in close contact with a region including at least a portion of a parietal lobe, occipital lobe, and temporal lobe of the subject.

8. The healthcare device for non-invasive brain stimulation according to claim 6, wherein some of the plurality of electrode units are installed at a location capable of adhering to a region of the first band including at least a portion of a parietal, occipital, and temporal lobes of the subject.

9. The healthcare device for non-invasive brain stimulation according to claim 6, further comprising a lower electrode adhesion module that is installed on the helmet,
wherein the lower electrode adhesion module includes:
a second band adjusting unit that is installed independently of the helmet; and
a second band of which length is adjusted by the second band adjusting unit and that is configured to partially cover a back of a neck or a back of an ear of the subject,
wherein some of the plurality of electrode units are installed on the second band, when the length of the second band is shortened by the second band adjusting unit, the electrode unit installed in the second band is in close contact with a region including at least a portion of the back of the ear, the back of the head, and the back of the neck of the subject.

10. The healthcare device for non-invasive brain stimulation according to claim 9, further comprising a sensor unit that is installed on the second band,
wherein when the length of the second band is shortened by the second band adjusting unit, the sensor unit is in close contact with a region including at least a part of the back of the ear, the back of the head, and the back of the neck of the subject.

11. The healthcare device for non-invasive brain stimulation according to claim 6, wherein the electrode units include a wet pad, and the helmet includes a first frame that is formed to cover from a forehead to a back of the head and has an opening at an upper portion thereof, and a second frame that extends upward from the first frame and crosses over the head of the subject, and humidity or moisture generated in the wet pad is discharged through the opening.

12. The healthcare device for non-invasive brain stimulation according to claim 6, wherein an LED monitoring unit that displays an operating state of the electrode units is installed on the helmet.
